(19) United States
(12) Patent Application Publication
(10) Pub. No.: US 2021/0353202 A1
(43) Pub. Date: Nov. 18, 2021

(54) ELECTROCARDIOGRAM TEST DEVICE AND ELECTROCARDIOGRAM TEST METHOD, AND NON-TRANSITORY COMPUTER READABLE MEDIUM THAT ARE TO BE USED IN THE DEVICE OR THE TEST METHOD

(71) Applicant: NIHON KOHDEN CORPORATION, Shinjuku-ku, Tokyo (JP)

(72) Inventor: Jiro SUTO, Tokorozawa-shi, Saitama (JP)

(21) Appl. No.: 17/287,321

(22) PCT Filed: Oct. 4, 2019
(86) PCT No.: PCT/JP2019/039290

§ 371 Date: Apr. 21, 2021

(30) Foreign Application Priority Data
Oct. 22, 2018 (JP) 2018-198225

(51) Int. Cl. A61B 5/327; A61B 5/00

(52) U.S. Cl. CPC A61B 5/327; A61B 5/742

(57) ABSTRACT

A non-transitory computer-readable medium having stored a computer program causes a computer to realize the functions of: receiving electrocardiogram waveform data from first electrodes attached to a subject, the data indicating a plurality of electrocardiogram waveforms that are continuously generated on a time axis; storing the electrocardiogram waveform data in a storage section; calculating coefficients for deriving estimated electrocardiogram waveform data of the subject, from first electrocardiogram waveform data that are stored in the storage section; and deriving the estimated electrocardiogram waveform data of the subject, based on second electrocardiogram waveform data that are acquired after the first electrocardiogram waveform data are stored and the coefficients.

### TECHNICAL FIELD

The presently disclosed subject matter relates to an electrocardiogram test device and an electrocardiogram test method, and a non-transitory computer readable medium that are to be used in the device or the test method.

### BACKGROUND ART

An electrocardiogram test that uses a standard 12 lead electrocardiogram is known. When an electrocardiogram test is to be performed by using a standard 12 lead electrocardiogram, four electrodes are attached to the four limbs of a subject, and six electrodes to the chest (see Patent Literature 1).

### CITATION LIST

### Patent Literature

[PTL 1] Japanese Patent Publication 2004-505658A

### SUMMARY OF INVENTION

### Technical Problem

In an electrocardiogram test, however, there is a case where a part of electrodes attached to the subject are detached from the subject. In the case where an electrode attached to the subject is detached during the test, electrocardiogram data that are obtained during the detachment cannot be used in the test. As a result, the electrocardiogram test must be sometimes redone. When, in an electrocardiogram test, electrodes are to be attached to a subject who has undergone open chest surgery, or a baby subject, moreover, there is a case where all of electrodes that are to be attached to the subject cannot be attached to the subject.

An illustrative aspect of the presently disclosed subject matter provides an electrocardiogram test device and electrocardiogram test method in which, even in the case where a part of electrodes that are to be attached to a subject are not attached, lead data (electrocardiogram waveform data) that cannot be directly measured are complemented from electrocardiogram waveform data that can be measured, whereby the reliability of an electrocardiogram test can be improved, and also a non-transitory computer readable medium that are to be used in the device or the test method.

### Solution to Problem

A non-transitory computer-readable medium having stored a computer program of a mode of the presently disclosed subject matter causes a computer to realize the functions of: receiving electrocardiogram waveform data from first electrodes attached to a subject, the data indicating a plurality of electrocardiogram waveforms that are continuously generated on a time axis;

storing the electrocardiogram waveform data in a storage section;

calculating coefficients for deriving estimated electrocardiogram waveform data of the subject, from first electrocardiogram waveform data that are stored in the storage section; and

deriving the estimated electrocardiogram waveform data of the subject, based on second electrocardiogram waveform data that are acquired after the first electrocardiogram waveform data are stored and the coefficients.

According to the non-transitory computer-readable medium having the above-described configuration, the coefficients for deriving the estimated electrocardiogram waveform data of the subject are calculated from the first electrocardiogram waveform data that are stored after the reception from the electrodes. The estimated electrocardiogram waveform data are derived based on the calculated coefficients and the second electrocardiogram waveform data.

According to the above-described configuration, therefore, it is possible to provide a program in which, even in the case where a part of the electrodes that are to be attached to the subject are not attached, second electrocardiogram waveform data that cannot be directly measured are complemented from second electrocardiogram waveform data that can be measured, whereby the reliability of the electrocardiogram test can be improved.

An electrocardiogram test method of a mode of the presently disclosed subject matter includes the steps of receiving electrocardiogram waveform data from first electrodes attached to a subject, the data indicating a plurality of electrocardiogram waveforms that are continuously generated on a time axis;

storing the received electrocardiogram waveform data;

calculating coefficients for deriving estimated electrocardiogram waveform data of the subject, from first electrocardiogram waveform data that are stored; and

deriving the estimated electrocardiogram waveform data of the subject, based on second electrocardiogram waveform data that are acquired after the first electrocardiogram waveform data are stored and the coefficients.

According to the electrocardiogram test method having the above-described configuration, the coefficients for deriving the estimated electrocardiogram waveform data of the subject are calculated from the first electrocardiogram waveform data that are stored after the reception from the electrodes. The estimated electrocardiogram waveform data are derived based on the coefficients and the second electrocardiogram waveform data.

According to the above-described configuration, therefore, it is possible to provide an electrocardiogram test method in which, even in the case where a part of the electrodes that are to be attached to the subject are not attached, second electrocardiogram waveform data that cannot be directly measured are complemented from second electrocardiogram waveform data that can be measured, whereby the reliability of the electrocardiogram test can be improved.

An electrocardiogram test device of a mode of the presently disclosed subject matter includes:

a receiver adapted to receive electrocardiogram waveform data from first electrodes attached to a subject, the data indicating a plurality of electrocardiogram waveforms that are continuously generated on a time axis;

a storage section adapted to store the electrocardiogram waveform data;

a calculating section adapted to calculate coefficients for deriving estimated electrocardiogram waveform data of the subject, from first electrocardiogram waveform data that are stored in the storage section; and

a deriving section adapted to derive the estimated electrocardiogram waveform data of the subject, based on second electrocardiogram waveform data that are acquired after the first electrocardiogram waveform data are stored and the coefficients.

According to the electrocardiogram test device having the above-described configuration, the coefficients for deriving the estimated electrocardiogram waveform data of the subject are calculated from the first electrocardiogram waveform data that are stored in the storage section after the receiver receives the data from the electrodes. The deriving section can derive the estimated electrocardiogram waveform data based on the calculated coefficients and the second electrocardiogram waveform data.

According to the above-described configuration, therefore, it is possible to provide an electrocardiogram test device in which, even in the case where a part of the electrodes that are to be attached to the subject are not attached, second electrocardiogram waveform data that cannot be directly measured are complemented from second electrocardiogram waveform data that can be measured, whereby the reliability of the electrocardiogram test can be improved.

### BRIEF DESCRIPTION OF DRAWINGS

FIG. 1 is a functional block diagram of an electrocardiogram test device 1 of an embodiment of the presently disclosed subject matter.

FIG. 2 is a functional block diagram of a controller of the electrocardiogram test device of FIG. 1.

FIG. 3 illustrates attachment positions of electrodes that are to be attached to the subject when the standard 12 lead system is used.

FIG. 4 is a flowchart illustrating the flow of a first operation example.

FIG. 5 is a flowchart illustrating the flow of a second operation example.

FIG. 6 is a flowchart illustrating the flow of a third operation example.

FIG. 7 illustrates an example of a screen that is displayed on a displaying section.

FIG. 8 illustrates an example of the screen that is displayed on the displaying section.

FIG. 9 illustrates an example of the screen that is displayed on the displaying section.

FIG. 10 illustrates an example of the screen that is displayed on the displaying section.

### DESCRIPTION OF EMBODIMENTS

Hereinafter, an embodiment of the presently disclosed subject matter will be described in detail with reference to the accompanying drawings. In the description of the embodiment, with respect to components that are denoted by the same reference numerals identifying components which have been described, their description is omitted for the sake of convenience in description.

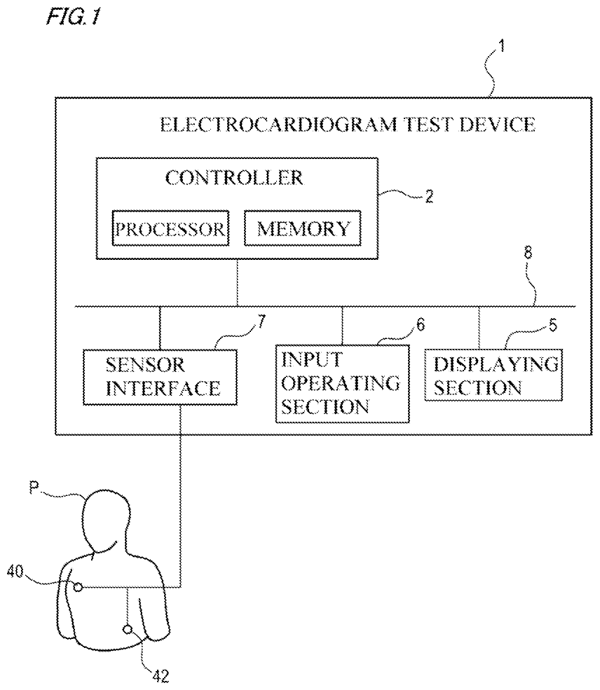

FIG. 1 is a functional block diagram of an electrocardiogram test device 1 of an embodiment of the presently disclosed subject matter (hereinafter, referred to merely as “the embodiment”). As illustrated in FIG. 1, the electrocardiogram test device 1 includes a controller 2, a displaying section 5, an input operating section 6, and a sensor interface 7. These components are communicably connected to one another through a bus 8. The electrocardiogram test device 1 includes a power source, digital/analog circuits, and the like that are not illustrated.

The controller 2 includes a memory and a processor. The memory may be configured by, for example, a ROM (Read Only Memory) that stores various programs and the like, and a RAM (Random Access Memory) having a plurality of work areas in which various programs to be executed by the processor, and the like are stored. For example, the processor is a CPU (Central Processing Unit), and configured so as to develop a designated one of the various programs installed in the ROM, in the RAM, and execute various processes (such as processes of functional blocks that will be described later) in cooperation with the RAM.

In the embodiment, a computer readable medium may be used. A computer readable medium means all kinds of physical memories (a RAM, a ROM, and the like) that can store information and data which can be read by a processor. The computer readable medium can store instructions relating to processes to be executed by one or more processors. It should be understood that the teen “computer readable medium” includes tangible items, and excludes a carrier wave and a transitory signal (namely, indicates a non-transitory medium).

The processor may load an electrocardiogram analysis program into the RAM, and execute the program in cooperation with the RAM, thereby enabling the controller 2 to control various operations of the electrocardiogram test device 1. A part of the processes of the controller 2 may be realized by various electric circuits that are disposed in the device. When the controller 2 receives no electrocardiogram waveform data from the sensor interface 7, or a measurement time period that is arbitrarily set elapses, the controller 2 determines that the measurement of electrocardiogram data is ended.

The displaying section 5 is a display device such as a liquid crystal display or an organic EL display. The displaying section 5 is configured so as to display a plurality of electrocardiogram waveforms that are continuously generated on a time axis, based on electrocardiogram waveform data that are acquired through the sensor interface 7, and estimated electrocardiogram waveform data that will be described later. The displaying section 5 may not be disposed in the electrocardiogram test device 1, and may have a configuration in which the section is wiredly or wirelessly connected with the electrocardiogram test device 1.

The input operating section 6 is configured so as to receive an input operation performed by an operator who operates the electrocardiogram test device 1, and produce an instruction signal corresponding to the input operation. The input operating section 6 is, for example, a touch panel that is overlaid on the displaying section 5, operation buttons mounted on the housing, or event switches. The input operating section 6 receives, for example, an input operation for starting the acquisition of electrocardiogram waveform data, or switching displayed waveforms, and produces an instruction signal corresponding to the input operation. The produced instruction signal is transmitted to the controller 2 through the bus 8. The controller 2 is configured so as to control the operation of the electrocardiogram test device 1 based on the instruction signal.

The sensor interface 7 is connected to electrodes 40 (40a to 40j) (one sample of first electrodes) attached to a subject P, electrodes 42 (one sample of second electrodes), and indifferent electrodes that are not illustrated. The electrodes 40 are attached to positions (hereinafter, referred to as normal positions) where, in the standard 12 lead system, electrodes are usually attached. By contrast, the electrodes 42 are attached to positions (hereinafter, referred to as non-normal positions) other than the positions where, in the standard 12 lead system, electrodes are usually attached. The electrodes 40 and the electrodes 42 are contacted to measurement portions of the subject P, and function as sensors for deriving potential changes of the measurement portions. The electrodes 40 and the electrodes 42 are configured so as to derive potential changes of the measurement portions, and the indifferent electrodes are configured so as to eliminate external noises that are induced in phase in the electrodes 40 and the electrodes 42.

Electrical signals indicating potential changes (potentials) that are output from the electrodes 40, those indicating potential changes (potentials) that are output from the electrodes 42, and those indicating potential changes (potentials) that are output from the indifferent electrodes are input to the sensor interface 7. The sensor interface 7 differentially amplifies the potentials that are derived from the electrodes 40 and/or those that are derived from the electrodes 42, eliminates external noises by using the potentials that are derived from the indifferent electrodes, and produces amplified electrocardiogram signals. Moreover, the sensor interface 7 analog-digital converts (AD converts) the amplified electrocardiogram signals to produce electrocardiogram waveform data. The produced electrocardiogram waveform data are supplied to the memory of the controller 2 through the bus 8. Here, the electrocardiogram waveform data indicate a plurality of electrocardiogram waveforms (waveforms that are produced by one beat) that are continuously generated on the time axis. In the specification, electrocardiogram waveform data that are initially acquired are referred to as first electrocardiogram waveform data, and electrocardiogram waveform data that are acquired after the first electrocardiogram waveform data are stored are referred to as second electrocardiogram waveform data. In the specification, unless otherwise indicated, the term “electrocardiogram waveform data” contains the first electrocardiogram waveform data and the second electrocardiogram waveform data. Moreover, the term “first electrocardiogram waveform data” contains third electrocardiogram waveform data and fourth electrocardiogram waveform data. The third electrocardiogram waveform data are data that are acquired in advance of the fourth second electrocardiogram waveform data.

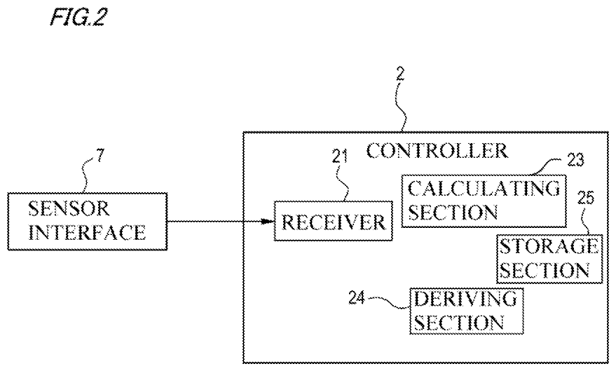

Next, the functional blocks of the controller 2 of the electrocardiogram test device 1 will be described with reference to FIG. 2. FIG. 2 illustrates the functional blocks of the controller 2 of the electrocardiogram test device 1. As illustrated in FIG. 2, the controller 2 may include a receiver 21, a calculating section 23, a deriving section 24, and a storage section 25.

The receiver 21 is configured so as to receive the electrocardiogram waveform data from the electrodes 40 attached to the subject P, through the sensor interface 7.

The calculating section 23 is configured so as to calculate coefficients for deriving the estimated electrocardiogram waveform data of the subject P, from the first electrocardiogram waveform data that are stored in the storage section 25. Examples of the method for calculating the coefficients are the multiple regression analysis method and the multivariate analysis.

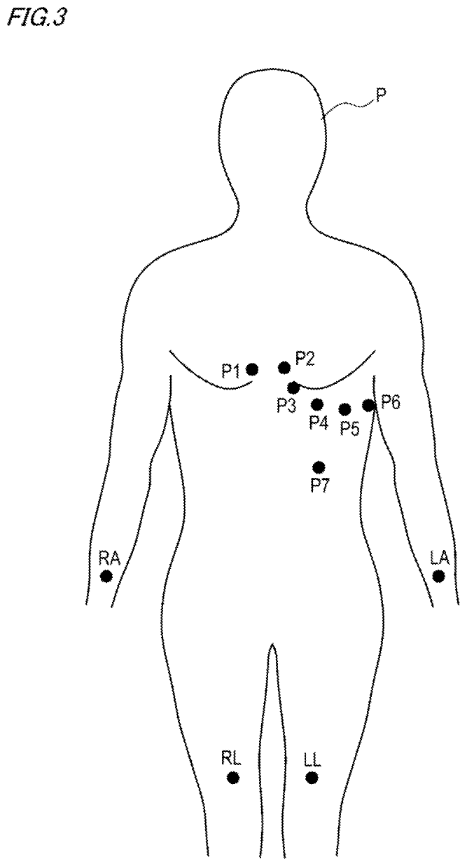

FIG. 3 illustrates attachment positions of the electrodes that are to be attached to the subject P when the standard 12 lead system is used. In the case where an electrocardiogram test is to be performed by using the standard 12 lead system, the electrodes 40a to 40f are attached to chest portions P1 to P6, and the electrodes 40g to 40j to the four limbs RA, LA, RL, and LL. The electrode 40a is attached to the position of the chest portion P1. The electrode 40b is attached to the position of the chest portion P2. The electrode 40c is attached to the position of the chest portion P3. The electrode 40d is attached to the position of the chest portion P4. The electrode 40e is attached to the position of the chest portion P5. The electrode 40f is attached to the position of the chest portion P6. The electrode 40g is attached to the position of the right arm RA. The electrode 40h is attached to the position of the left arm LA. The electrode 40i is attached to the position of the right leg RL. The electrode 40j is attached to the position of the left leg LL. The chest portions P1 to P6 and the four limbs RA, LA, RL, and LL are the normal positions. Portions other than the ten portions, for example, the portion P7 is a non-normal position.

In the case an electrocardiogram test is to be performed by using the standard 12 lead system, leads I to III, lead aVR, lead aVL, lead aVF, and leads V1 to V6 are employed. In lead I, the anode is in the left arm LA, and the cathode is in the right arm RA. In lead II, the anode is in the left leg LL, and the cathode is in the right arm RA. In lead III, the anode is in the left leg LL, and the cathode is in the left arm LA. In lead aVR, the anode is in the right arm RA, and the cathode is in the midpoint M1 between the left arm LA and the left leg LL. In lead aVL, the anode is in the left arm LA, and the cathode is in the midpoint M2 between the right arm RA and the left leg LL. In lead aVF, the anode is in the left leg LL, and the cathode is in the midpoint M3 between the right arm RA and the left arm LA. In lead V1, the anode is the chest portion P1, and the cathode is the midpoint M4 among the right arm RA, the left arm LA, and the left leg LL. In lead V2, the anode is in the chest portion P2, and the cathode is in the midpoint M4. In lead V3, the anode is the chest portion P3, and the cathode is the midpoint M4. In lead V4, the anode is in the chest portion P4, and the cathode is in the midpoint M4. In lead V5, the anode is in the chest portion P5, and the cathode is in the midpoint M4. In lead V6, the anode is in the chest portion P6, and the cathode is in the midpoint M4.

Next, the way how the estimated electrocardiogram waveform data in lead V4 is derived in the case where the electrode cannot be attached to the position of the chest portion P4 will be described.

First, coefficients β₀ to β₇ satisfying following Expression (1) are obtained from a group of electrocardiograms that are actually measured.

αV₄=β₀+β₁×V₁+β₂×V_{I1}+β₃×V_{V1}+β₄×V_{V2}+β₅×V_{V3}+β₆×V_{V5}+β₇×V_{V6} [Math. 1]

In the above expression, V₁ indicates electrocardiogram waveform data in lead I, V_{II} indicates electrocardiogram waveform data in lead II, V_{V1} indicates electrocardiogram waveform data in lead V1, V_{V2} indicates electrocardiogram waveform data in lead V2, V_{V3} indicates electrocardiogram waveform data in lead V3, V_{V5} indicates electrocardiogram waveform data in lead V5, V_{V6} indicates electrocardiogram waveform data in lead V6, αV₄ indicates electrocardiogram waveform data in lead V4 that are obtained in an actual measurement, β₀ is a number for adjusting the error in order to satisfy Expression (1), β₁ is a coefficient in lead I, β₂ is a coefficient in lead II, β₃ is a coefficient in lead V1, β₄ is a coefficient in lead V2, β₅ is a coefficient in lead V3, β₆ is a coefficient in lead V5, and β₇ is a coefficient in lead V6. In Expression (1), V_{I}, V_{II}, V_{V2}, V_{V2}, V_{V3}, V_{V5}, V_{V6}, and αV₄ indicate the first electrocardiogram waveform data.

From the coefficients 13o to P7 that are obtained in Expression (1) above, thereafter, the estimated electrocardiogram waveform data in lead V4 are obtained by using following Expression (2). In Expression (2), V_{I}, V_{II}, V_{V1}, V_{V2}, V_{V3}, V_{V5}, and V_{V6} indicate the second electrocardiogram waveform data. Moreover, a′V₄ indicates the estimated electrocardiogram waveform data in lead V4.

α′V₄=β₀+β₁/V₁β₂×V_{I1}+β₃×V_{V1}+β₄×V_{V2}+β₅×V_{V3}+β₆×V_{V5}+β₇×V_{V6} [Math. 2]

Returning to FIG. 2, the deriving section 24 is configured so as to derive the estimated electrocardiogram waveform data of the subject P based on the second electrocardiogram waveform data that are stored in the storage section 25, and the coefficients that are calculated by the calculating section 23. The estimated electrocardiogram waveform data are estimated values of electrocardiogram waveform data that are derived from the second electrocardiogram waveform data obtained from the electrodes 40 or electrodes 42 which are attached to the subject P, and the coefficients, and that are in the normal positions to which the electrodes 40 are not attached.

The storage section 25 can store electrocardiogram waveform data, the coefficients calculated by the calculating section 23, the estimated electrocardiogram waveform data derived by the deriving section 24, etc.

### First Operation Example

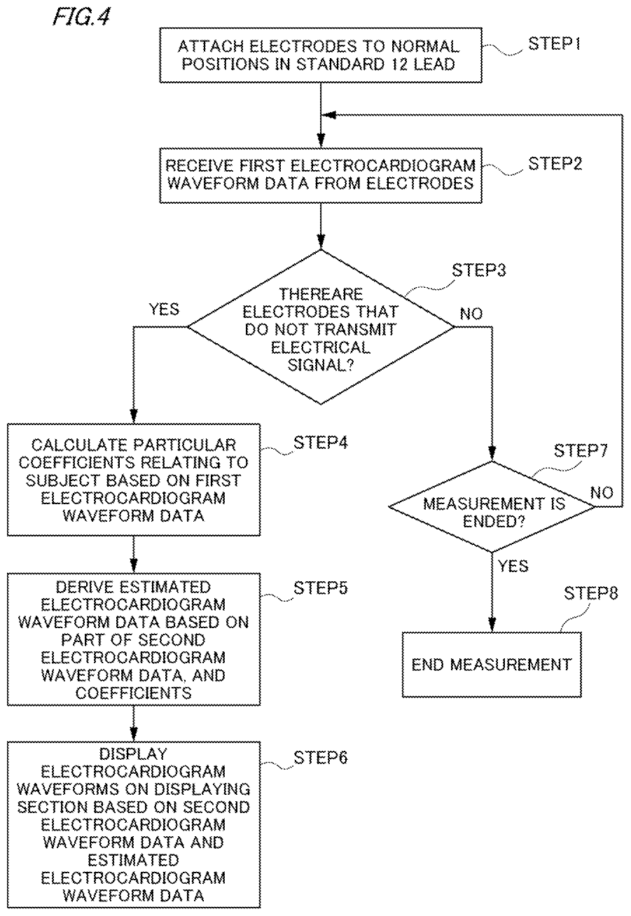

FIG. 4 is a flowchart illustrating the flow of a first operation example. For example, the first operation example is performed in the case where, during an electrocardiogram test, a part of the electrodes 40 attached to the subject P are detached. In the case where an electrocardiogram test is to be performed by using the standard 12 lead system, a medical person attaches the electrodes 40 to the normal positions of the subject P (STEP1). The electrodes 40 transmit the electrical signals indicating potential changes (potentials) to the sensor interface 7. The sensor interface 7 produces the electrocardiogram signals based on the electrical signals. The sensor interface 7 further produces the first electrocardiogram waveform data based on the electrocardiogram signals.

The sensor interface 7 transmits the first electrocardiogram waveform data to the receiver 21 of the controller 2 (STEP2). The first electrocardiogram waveform data are stored in the storage section 25. A plurality of electrocardiogram waveforms that are continuously generated on the time axis are displayed on the displaying section 5 based on the first electrocardiogram waveform data.

After STEP2, if there are electrodes 40 that do not transmit an electrical signal to the sensor interface 7 (YES in STEP3), the sensor interface 7 produces the electrocardiogram signals based on the electrical signals that are received from the electrodes 40, and produces the second electrocardiogram waveform data based on the electrocardiogram signals. The calculating section 23 of the controller 2 calculates the coefficients for deriving the estimated electrocardiogram waveform data of the subject P, from the first electrocardiogram waveform data that are stored in the storage section 25 (STEP4). The calculated coefficients are stored in the storage section 25. Then, the deriving section 24 derives the estimated electrocardiogram waveform data based on the second electrocardiogram waveform data and coefficients that are stored in the storage section 25 (STEP5). The estimated electrocardiogram waveform data are stored in the storage section 25. The term of the second electrocardiogram waveform data here means electrocardiogram waveform data that are acquired after appearance of the electrodes 40 which do not transmit an electrical signal to the sensor interface 7. Namely, the second electrocardiogram waveform data are electrocardiogram waveform data that are acquired after the first electrocardiogram waveform data are stored.

A plurality of electrocardiogram waveforms that are continuously generated on the time axis based on the electrocardiogram waveform data and the estimated electrocardiogram waveform data are displayed on the displaying section 5 (STEP6). When an electrical signal is not transmitted from any the electrodes 40 attached to the subject P, to the sensor interface 7, or when a measurement time period that is arbitrarily set elapses, the measurement of electrocardiogram waveform data is ended. By contrast, if there is no electrodes 40 that do not transmit an electrical signal to the sensor interface 7 (NO in STEP3), the controller 2 determines whether the measurement of the first electrocardiogram waveform data is ended or not (STEP7). If an electrical signal is not transmitted from any the electrodes 40 attached to the subject P, to the sensor interface 7, and the electrocardiogram waveform data are not transmitted to the receiver 21 of the controller 2, for example, the controller 2 determines that the measurement of the first electrocardiogram waveform data is ended. If the measurement of the electrocardiogram waveform data is ended (YES in STEP7), the measurement of the electrocardiogram waveform data is ended (STEP8). By contrast, if the measurement of the electrocardiogram waveform data is not ended (NO in STEP7), the process returns to STEP2.

STEP4 may be executed before the execution of STEP3. That is, the calculation of particular coefficients relating to the subject that is based on the first electrocardiogram waveform data may be performed before the determination whether there are the electrodes 40 that do not transmit an electrical signal to the sensor interface 7 or not. The calculation of the coefficients may be performed after the end of the measurement of all of the second electrocardiogram waveform data.

### Second Operation Example

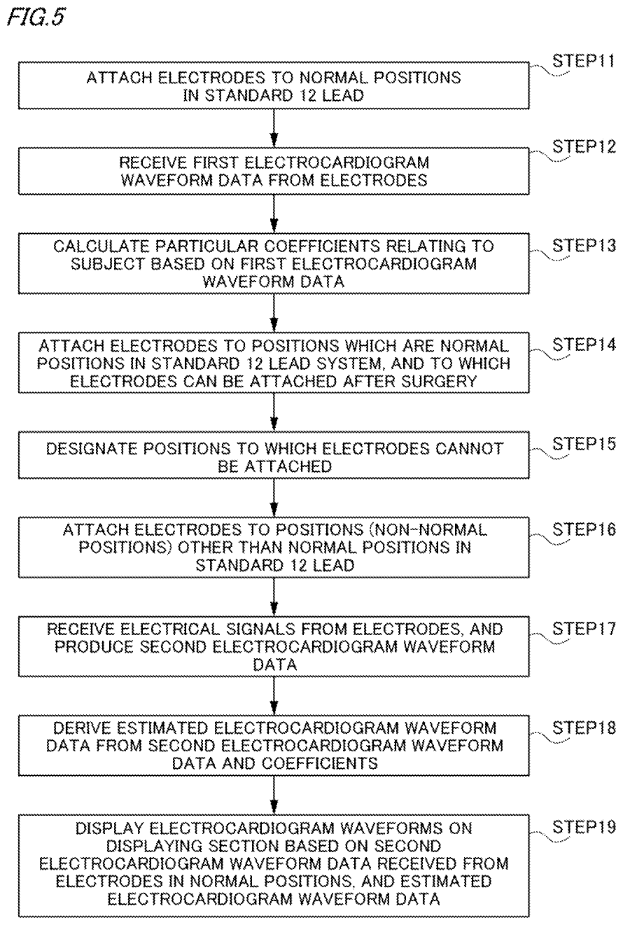

FIG. 5 is a flowchart illustrating the flow of a second operation example. For example, the second operation example is performed in the case where the subject P has undergone cardiac surgery, and therefore the electrodes 40 cannot be attached to a part of the normal positions. In the specification, the second operation example will be described by using this example. STEP11 to STEP13 are same or similar to STEP1, STEP2, and STEP4 of the first operation example, and hence their description is omitted. STEP11 to STEP13 are usually performed before the surgery.

In the case where the subject P undergoes cardiac surgery, and therefore the medical person cannot attach the electrodes 40 to a part of the normal positions, for example, the medical person attaches the electrodes 40 to only the normal positions of the subject P to which the electrodes 40 can be attached, after the coefficients are calculated (STEP14). The medical person inputs information of the normal positions to which the electrodes 40 cannot be attached, through the input operating section 6 (STEP′ 5). The medical person attaches the electrodes 42 to arbitrary non-normal positions of the subject P, in place of the normal positions to which the electrodes 40 cannot be attached (STEP16). Preferably, the non-normal positions are close to the normal positions to which the electrodes 40 cannot be attached.

The sensor interface 7 produces the electrocardiogram signals based on the electrical signals that are received from the electrodes 40 and the electrodes 42. The sensor interface 7 further produces the second electrocardiogram waveform data based on the electrocardiogram signals (STEP17). The second electrocardiogram waveform data are stored in the storage section 25.

The deriving section 24 derives the estimated electrocardiogram waveform data based on the second electrocardiogram waveform data and the coefficients (STEP18). The estimated electrocardiogram waveform data are stored in the storage section 25. STEP19 is same or similar to STEP6 of the first operation example, and hence its description is omitted.

Alternatively, STEP13 may be executed after STEP17 is executed. The calculation of particular coefficients relating to the subject that is based on the first electrocardiogram waveform data may be performed in parallel with the measurement of the second electrocardiogram waveform data, or performed after the end of the measurement of all of the second electrocardiogram waveform data.

### Third Operation Example

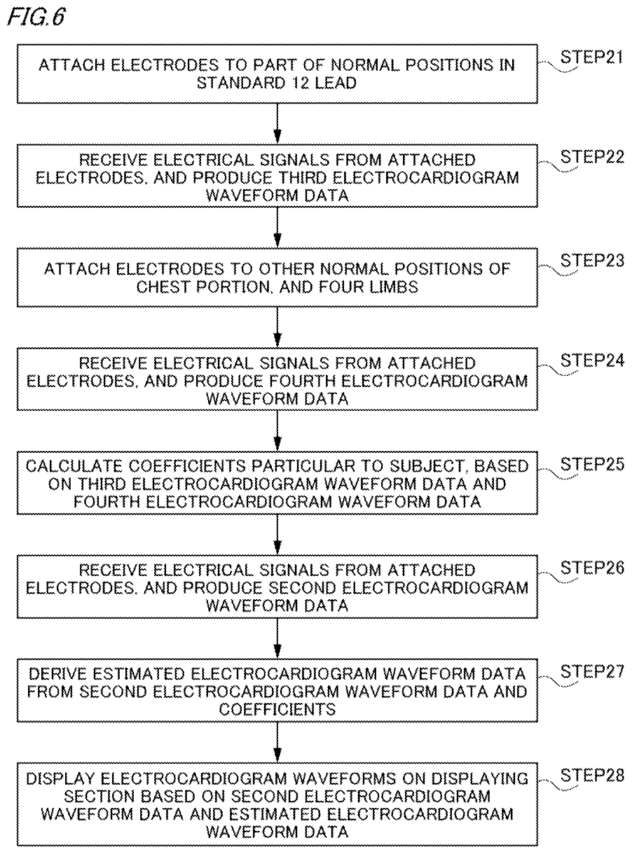

FIG. 6 is a flowchart illustrating the flow of a third operation example. For example, the third operation example is performed in the case where the subject P is a baby in whom the electrodes 40 can be attached only to a part of the normal positions.

The medical person attaches the electrodes 40 to the part of normal positions of the subject P (STEP21). For example, the medical person attaches the electrode 40a to the chest portion P1, the electrode 40c to the chest portion P3, the electrode 40e to the chest portion P5, and the electrodes 40g to 40j to the four limbs RA, LA, RL, and LL.

The sensor interface 7 produces the electrocardiogram signals based on the electrical signals that are received from the attached electrodes 40. The sensor interface 7 further produces the first electrocardiogram waveform data based on the electrocardiogram signals. However, the first electrocardiogram waveform data are third electrocardiogram waveform data that are produced based on the electrocardiogram signals based on the electrical signals which are received from a part of the electrodes 40 (in the example, the electrode 40a, the electrode 40c, the electrode 40e, and the electrodes 40g to 40j) (STEP22). The third electrocardiogram waveform data are transmitted to the storage section 25.

The medical person detaches the electrode 40a, the electrode 40c, and the electrode 40e from the subject P. The medical person attaches the electrode 40b to the chest portion P2 of the subject P, the electrode 40d to the chest portion P4, the electrode 40f to the chest portion P6, and the electrodes 40g to 40j to the four limbs (STEP23).

The sensor interface 7 produces the electrocardiogram signals based on the electrical signals that are received from the electrodes. The sensor interface 7 further produces fourth electrocardiogram waveform data based on the electrocardiogram signals (STEP24). The fourth electrocardiogram waveform data are transmitted to the storage section 25 of the controller 2.

The calculating section 23 of the controller 2 calculates the coefficients for deriving the estimated electrocardiogram waveform data of the subject P, based on the third electrocardiogram waveform data and the fourth electrocardiogram waveform data (STEP25). The calculated coefficients are stored in the storage section 25.

Here, the electrode 40b, the electrode 40d, the electrode 40f, and the electrodes 40g to 40j are continued to be attached to the subject P. The sensor interface 7 produces electrocardiogram signals based on the electrical signals that are received from the electrode 40b, the electrode 40d, the electrode 40f, and the electrodes 40g to 40j. The sensor interface 7 produces the second electrocardiogram waveform data based on the electrocardiogram signals (STEP26). The second electrocardiogram waveform data are transmitted to the storage section 25 of the controller 2.

Based on the second electrocardiogram waveform data and the coefficients, the deriving section 24 derives the estimated electrocardiogram waveform data in the normal positions to which the electrodes 40 are not attached (in the example, the positions to which the electrodes 40a, 40c, and 40e are applied) (STEP27). The estimated electrocardiogram waveform data are transmitted to the storage section 25 of the controller 2. STEP28 is same or similar to STEP6 of the first operation example, and hence its description is omitted.

Alternatively, STEP25 may be executed after STEP26 is executed. The calculation of particular coefficients relating to the subject that is based on the first electrocardiogram waveform data may be performed in parallel with the measurement of the second electrocardiogram waveform data, or performed after the end of the measurement of all of the second electrocardiogram waveform data.

### Display Example

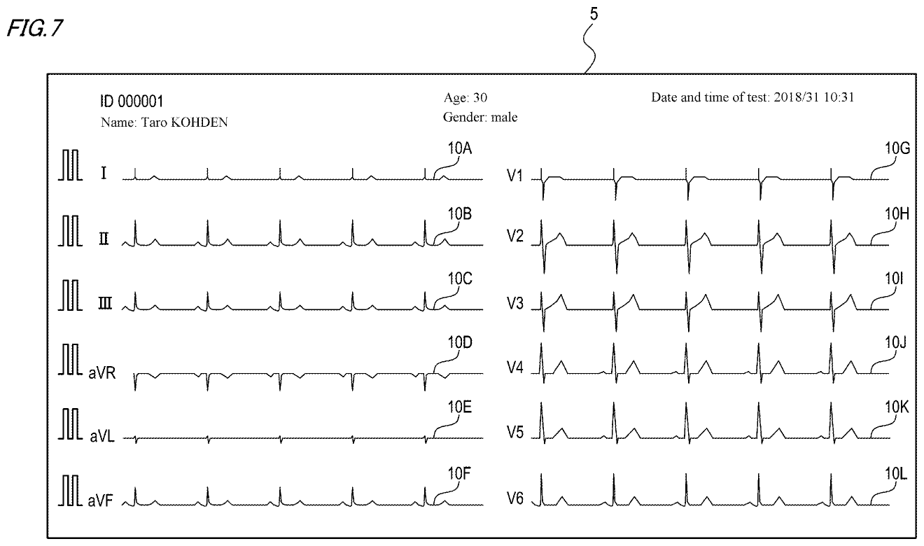

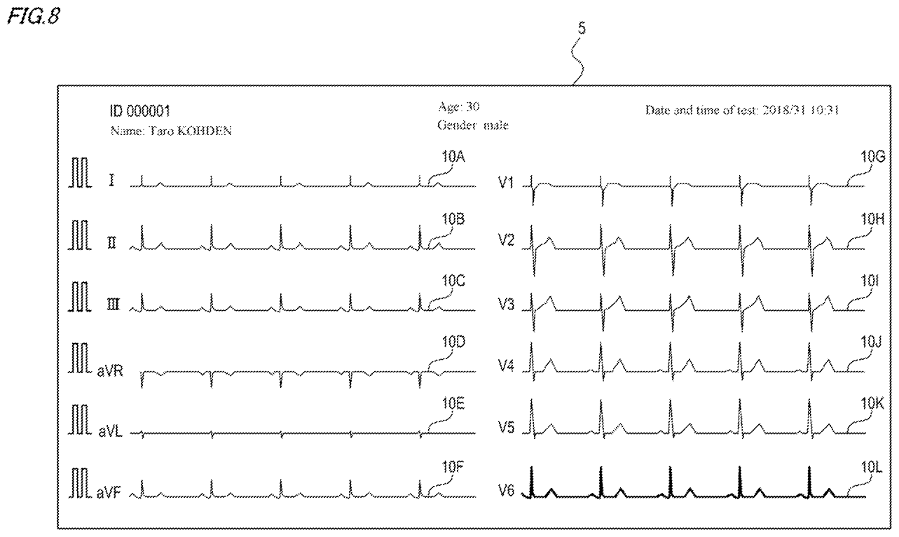

FIG. 7 illustrates an example of a screen that is displayed on the displaying section 5. The controller 2 controls the displaying section 5 so as to display electrocardiogram waveforms based on the electrocardiogram waveform data and estimated electrocardiogram waveform data that are stored in the storage section 25. As illustrated in FIG. 7, then, a plurality of electrocardiogram waveforms 10A to 10L that are continuously generated on the time axis are displayed on the displaying section 5. In the case where the electrocardiogram waveform 10L is an electrocardiogram waveform based on the estimated electrocardiogram waveform data, for example, the medical person may perform, through the input operating section 6, an input operation that causes the display of the electrocardiogram waveform 10L to be in a display mode which is different from the displays of the other electrocardiogram waveforms 10A to 10K. When the medical person performs this input operation through the input operating section 6, the input operating section 6 produces an instruction signal based on the operation signal. The instruction signal is transmitted to the controller 2 through the bus 8. Based on the instruction signal, the controller 2 controls the displaying section 5 so as to display the electrocardiogram waveform 10L with a line that is thicker than the other electrocardiogram waveforms 10A to 10K. As a result, the screen of the displaying section 5 is changed to the display mode illustrated in FIG. 8. The electrocardiogram waveforms that are displayed on the displaying section 5 may contain a plurality of electrocardiogram waveforms that are based on the estimated electrocardiogram waveform data. In this case, when the medical person performs the same or similar input operation through the input operating section 6, the electrocardiogram waveforms that are based on the estimated electrocardiogram waveform data are displayed with a line that is thicker than the other electrocardiogram waveforms. The method of changing the display is not limited to that in which the thickness of the line is changed. For example, hatching may be applied, or the waveforms may be colored with an arbitrary color.

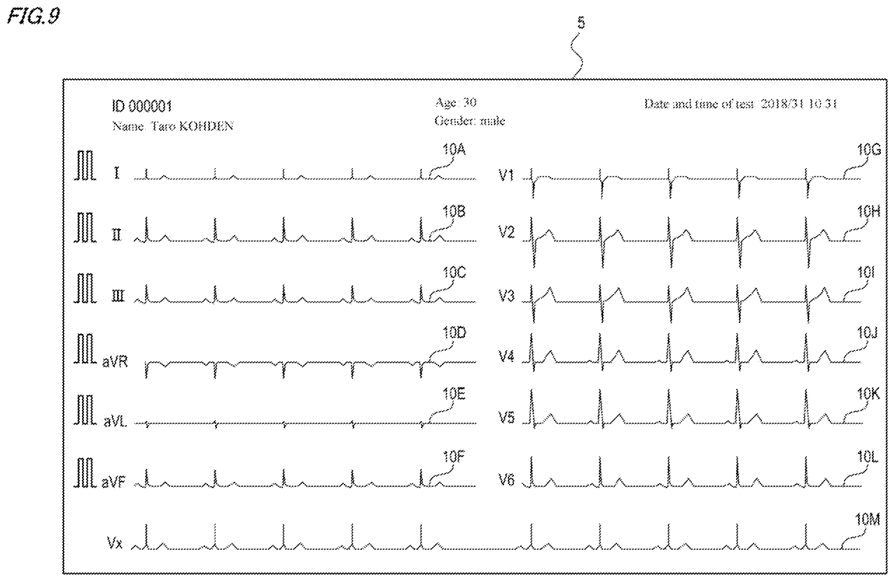

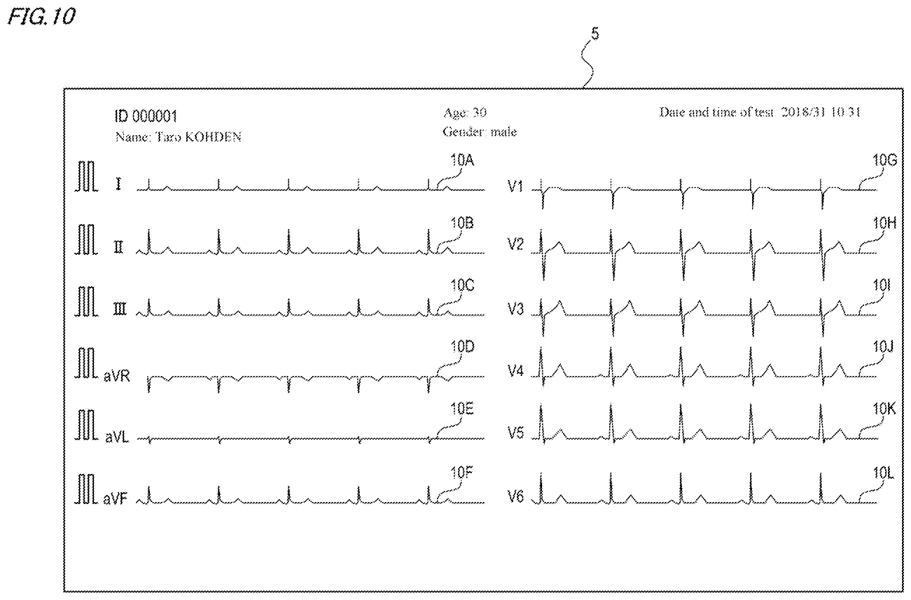

FIGS. 9 and 10 illustrate examples of the screen that is displayed on the displaying section 5. In the second operation example, the second electrocardiogram waveform data that are produced based on the electrical signals received from the electrodes 40, and those that are produced based on the electrical signals received from the electrodes 42 are stored in the storage section 25. In the case where the electrode 40 cannot be attached to the chest portion P4, and instead the electrode 42 is attached to the chest portion P7, for example, lead Vx can be derived while the anode is in the chest portion P7, and the cathode is in the midpoint M4. The medical person can perform, through the input operating section 6, an input operation for causing the displaying section 5 to display the electrocardiogram waveform 10M associated with lead Vx. When the medical person performs, through the input operating section 6, an input operation for causing the displaying section 5 to display the electrocardiogram waveform 10M, the displaying section 5 is controlled by the controller 2 so as to display a screen such as illustrated in FIG. 9. By contrast, when the medical person performs, through the input operating section 6, an input operation for causing the displaying section 5 not to display the electrocardiogram waveform 10M associated with lead Vx, the displaying section 5 is controlled by the controller 2 so as to display a screen such as illustrated in FIG. 10.

In an electrocardiogram test, in the case where a part of the electrodes 40 attached to the subject P are detached from the subject P, the medical person redoes the electrocardiogram test. In the case where all of the electrodes 40 that are to be attached to the subject P cannot be attached, moreover, the electrocardiogram test cannot be performed, or the manner of performing the electrocardiogram test is cumbersome.

In the non-transitory computer-readable medium having stored a computer program, electrocardiogram test method, and electrocardiogram test device 1 that are configured as described above, the coefficients for deriving the estimated electrocardiogram waveform data of the subject P are calculated from the first electrocardiogram waveform data that are stored after the data are received from the electrodes 40. Then, the estimated electrocardiogram waveform data are derived based on the calculated coefficients and the second electrocardiogram waveform data.

Even in the case where a part of the electrodes 40 that are to be attached to the subject are not attached, therefore, the second electrocardiogram waveform data that cannot be directly measured are complemented from the second electrocardiogram waveform data that can be measured, and the reliability of the electrocardiogram test can be improved.

According to the non-transitory computer-readable medium having stored a computer program, electrocardiogram test method, and electrocardiogram test device 1 that are configured as described above, in the first operation example, even when the second electrocardiogram waveform data cannot be acquired because of the phenomenon in which a part of the electrodes are detached from the subject P, the second electrocardiogram waveform data that cannot be directly measured are complemented from the second electrocardiogram waveform data that can be measured, and the reliability of the electrocardiogram test can be improved.

According to the non-transitory computer-readable medium having stored a computer program, electrocardiogram test method, and electrocardiogram test device 1 that are configured as described above, in the second operation example, the second electrocardiogram waveform data are usually received from not only the electrodes 40 that are attached to the subject P, but also the electrodes 42 other than the electrodes. Then, the estimated electrocardiogram waveform data are derived based on the second electrocardiogram waveform data and the coefficients.

According to the configuration, therefore, it is possible to improve the reliability of the electrocardiogram test even in the case where a part of the electrodes 40 which are to be attached to the subject P cannot be attached, complements the second electrocardiogram waveform data which cannot be directly measured, from the second electrocardiogram waveform data which can be measured.

According to the non-transitory computer-readable medium having stored a computer program, electrocardiogram test method, and electrocardiogram test device 1 that are configured as described above, in the third operation example, the coefficients can be derived from the third electrocardiogram waveform data that are received from a part of electrodes 40, and the fourth electrocardiogram waveform data that are received from the other part of electrodes 40 after the third electrocardiogram waveform data are received. According to the configuration, even in the case where a part of the electrodes 40 that are to be attached to the subject P cannot be attached, therefore, the estimated electrocardiogram waveform data can be derived.

According to the non-transitory computer-readable medium having stored a computer program, electrocardiogram test method, and electrocardiogram test device 1 that are configured as described above, an electrocardiogram waveform that is based on the estimated electrocardiogram waveform data is displayed in a display format which is different from that of the electrocardiogram waveform based on the electrocardiogram waveform data. Therefore, it is easy to distinguish between electrocardiogram waveforms that are based on the measured electrocardiogram waveforms, and an electrocardiogram waveform that is based on the estimated electrocardiogram waveform data.

According to the non-transitory computer-readable medium having stored a computer program, electrocardiogram test method, and electrocardiogram test device 1 that are configured as described above, in the standard 12 lead electrocardiogram test, only electrocardiogram waveforms that are based on the electrocardiogram waveform data which are received from the electrodes 40 that are attached to the subject P are usually displayed. Therefore, it is possible to prevent an excessively large number of electrocardiogram waveforms from being displayed on the displaying section 5.

The third operation example has been described by using the example in which the coefficients are calculated while the attachment of the electrodes 40 to the subject P is dividedly performed two times. However, the configuration is not limited to the example. The attachment of the electrodes 40 to the subject P may be dividedly performed three or more times.

The display example has been described by using the example in which, in order to obtain the different display modes, the medical person performs an input operation for differentiating the display mode, through the input operating section 6. However, the configuration is not limited to the example. A configuration may be employed where the controller 2 controls the displaying section 5, and the display mode is automatically changed.

The presently disclosed subject matter is not limited to the above-described embodiment, and may be freely subjected to modifications, improvements, and the like. In addition, the materials, shapes, dimensions, values, forms, numbers, installation places, and the like of the components of the above-described embodiment are arbitrary and not limited insofar as the presently disclosed subject matter can be achieved.

The present application is based on Japanese Patent Application No. 2018-198225 filed on Oct. 22, 2018, the entire contents of which are hereby incorporated by reference.

1. A non-transitory computer-readable medium having stored a computer program for causing a computer to realize the functions of:
receiving electrocardiogram waveform data from first electrodes attached to a subject, the data indicating a plurality of electrocardiogram waveforms that are continuously generated on a time axis;
storing the electrocardiogram waveform data in a storage section;
calculating coefficients for deriving estimated electrocardiogram waveform data of the subject, from first electrocardiogram waveform data that are stored in the storage section; and
deriving the estimated electrocardiogram waveform data of the subject, based on second electrocardiogram waveform data that are acquired after the first electrocardiogram waveform data are stored and the coefficients.

2. The non-transitory computer-readable medium according to claim 1, wherein,
in a case where there are electrodes from which the electrocardiogram waveform data are not acquired, the estimated electrocardiogram waveform data are derived based on:
the coefficients; and
the second electrocardiogram waveform data that are received from electrodes other than the electrodes from which the electrocardiogram waveform data are not acquired.

3. The non-transitory computer-readable medium according to claim 1, wherein the functions further includes a function of deriving the estimated electrocardiogram waveform data based on; the second electrocardiogram waveform data that are received from a second electrode(s) which is attached to a non-normal position(s) other than normal positions in which, in an electrocardiogram test, usual attachment to the subject is performed; and the coefficients.

4. The non-transitory computer-readable medium according to claim 1, wherein
the first electrocardiogram waveform data contains:
third electrocardiogram waveform data that are received from a part of the first electrodes; and
fourth electrocardiogram waveform data that are received from an electrode(s) other than the part of the first electrodes, and that are received after the third electrocardiogram waveform data are received, and
the coefficients are calculated based on the third electrocardiogram waveform data and the fourth electrocardiogram waveform data.

5. The non-transitory computer-readable medium according to claim 1, wherein the functions further includes a function of causing the electrocardiogram waveform that is based on the estimated electrocardiogram waveform data, to be displayed in a display format which is different from a display format of the electrocardiogram waveform based on the electrocardiogram waveform data.

6. The non-transitory computer-readable medium according to claim 3, wherein the functions further includes a function of causing the electrocardiogram waveform that is based on the second electrocardiogram waveform data which are received from the second electrode(s), not to be displayed.

7. An electrocardiogram test method including the steps of:
receiving electrocardiogram waveform data from first electrodes attached to a subject, the data indicating a plurality of electrocardiogram waveforms that are continuously generated on a time axis;
storing the received electrocardiogram waveform data;
calculating coefficients for deriving estimated electrocardiogram waveform data of the subject, from first electrocardiogram waveform data that are stored; and
deriving the estimated electrocardiogram waveform data of the subject, based on second electrocardiogram waveform data that are acquired after the first electrocardiogram waveform data are stored and the coefficients.

8. An electrocardiogram test device including:
a receiver adapted to receive electrocardiogram waveform data from first electrodes attached to a subject, the data indicating a plurality of electrocardiogram waveforms that are continuously generated on a time axis;
a storage section adapted to store the electrocardiogram waveform data;
a calculating section adapted to calculate coefficients for deriving estimated electrocardiogram waveform data of the subject, from first electrocardiogram waveform data that are stored in the storage section; and
a deriving section adapted to derive the estimated electrocardiogram waveform data of the subject, based on second electrocardiogram waveform data that are acquired after the first electrocardiogram waveform data are stored and the coefficients.
